(19) United States
(12) Patent Application Publication
(10) Pub. No.: US 2021/0214300 A1
(43) Pub. Date: Jul. 15, 2021

(54) ZEOLITE CATALYZED PROCESS FOR THE AMINATION OF ALKYLENE OXIDES

(71) Applicant: BASF SE, Ludwigshafen am Rhein (DE)

(72) Inventors: Andrei-Nicolae PARVULESCU, Ludwigshafen am Rhein (DE); Johann-Peter MELDER, Ludwigshafen am Rhein (DE); Ulrich MUELLER, Ludwigshafen am Rhein (DE); Alexander Michael HAYDL, Ludwigshafen am Rhein (DE); Zeljko KOTANJAC, Ludwigshafen am Rhein (DE); Hermann LUYKEN, Ludwigshafen am Rhein (DE); Christian GRUENANGER, Ludwigshafen am Rhein (DE)

(21) Appl. No.: 15/733,839

(22) PCT Filed: May 29, 2019
(86) PCT No.: PCT/EP2019/064030

§ 371 Date: Nov. 30, 2020

(30) Foreign Application Priority Data
May 30, 2018 (EP) 18175079.5

(51) Int. Cl. C07C 213/04; B01J 29/40; B01J 29/70; B01J 35/10; B01J 37/02; B01J 37/00; B01J 37/08

(52) U.S. Cl. CPC C07C 213/04; B01J 29/405; B01J 29/7049; B01J 35/1019; B01J 2229/186; B01J 37/0009; B01J 37/0201; B01J 37/082; B01J 37/0236

(57) ABSTRACT

The present invention relates to a process for the conversion of ethylene oxide to 2-aminoethanol and/or Di(2-hydroxyethyl)amine comprising (i) providing a catalyst comprising a zeolitic material comprising YO2 and X2O3 in its framework structure, wherein Y is a tetravalent element and X is a trivalent element, wherein the zeolitic material has a framework-type structure selected from the group consisting of MFI and/or MEL, including MEL/MFI intergrowths, and wherein the zeolitic material contains one or more rare earth elements; (ii) providing a mixture in the liquid phase comprising ethylene oxide and ammonia; (iii) contacting the catalyst provided in (i) with the mixture in the liquid phase provided in (ii) for converting ethylene oxide to 2-aminoethanol and/or Di(2-hydroxyethyl)amine, wherein the catalyst provided in (i) is obtained and/or obtainable by a process comprising loading one or more salts of the one or more rare earth elements into the pores of the porous structure of the zeolitic material and optionally on the surface of the zeolitic material.

### TECHNICAL FIELD

The present invention relates to a process for the conversion of ethylene oxide to 2-aminoethanol and/or Di(2-hydroxyethyl)amine using a rare earth element containing zeolite catalyst having a framework-type structure selected from the group consisting of MFI and/or MEL, including MEL/MFI intergrowths.

### INTRODUCTION

Alkanol amines are currently produced over a two/step process. In the first process step monoethanol amine is obtained through liquid phase amination of ethylene oxide (EO), wherein further amination steps eventually lead to a mixture of monoethanol amine (MEOA), diethanol amine (DEOA) and triethanol amine (TEOA). Depending on the molar ratio of ammonia to ethylene oxide, the distribution can be controlled to a certain extent. Since the demand for MEOA is however expected to grow stronger than the DEOA demand, it is therefore desirable to provide a process with a higher selectivity towards MEOA.

DE 1941859 and U.S. Pat. No. 3,697,598 respectively concern the reaction of ethylene oxide with ammonia over an acidic cation exchange resin as catalyst. U.S. Pat. No. 4,438,281, on the other hand, concerns the production of monoalkanolamines from alkylene oxides and ammonia over acidic inorganic catalysts such as acidic silica-aluminas, natural zeolites, and acid clays, amongst others. EP 0375267 A2 relates to the preparation of monoalkanolamines from ammonia and alkylene oxide over acid modified montmorillonite clay as a catalyst.

CN 101884934 relates to a molecular sieve catalyst for producing ethanolamine from ethylene oxide and ammonia using a ZSM-5 catalyst which has been surface modified with tetraethoxysilane. Feng, R. et al. in Catalysis Communications 2010, 11, pp. 1220-1223 describe the amination of ethylene oxide over HZSM-5, wherein the different catalysts used have been treated with EDTA, with tetraethyl orthosilicate, or have been prepared with varying silica to alumina ratios.

EP 1 104 752 A2 concerns a method of producing alkanolamines and apparatus for producing same.

JP 2002 028492 A concerns a producing method of diakanolamine, catalyst for producing dialkanolamine and producing method thereof.

EP 1 219 592 A1 concerns a method for production of alkanolamine and apparatus therefore. Marceau, E. et al. in “Ion Exchange and Impregnation: “Handbook of heterogeneous catalysis” (1972), vol. 107, pages 467-484 relates to ion exchange and impregnation.

Finally, U.S. Pat. Nos. 5,599,999 and 6,169,207 B1 respectively relate to a process for the preparation of alkanolamines from an alkylene oxide and ammonia aver a catalyst comprising a rare earth element supported on a carrier which may be a zeolite. In specific examples of said documents, lanthanum supported on ZSM-5 is employed as the catalyst, wherein lanthanum is ion exchanged into the zeolitic material, and wherein the catalyst is then employed for the amination of ethylene oxide with ammonia, respectively. According to the examples of U.S. Pat. No. 5,599,999, ion exchange of ZSM-5 with lanthanum nitrate would afford a catalyst containing 10 wt.-% of lanthanum calculated as the element. As demonstrated in the experimental section of the present application (see Comparative Example 5), however, repetition of the procedure of U.S. Pat. No. 5,599,999 affords a loading of 1 wt.-% of lanthanum calculated as the element, such that the disclosure obviously contains a typo with regard to the loading of lanthanum disclosed therein.

Despite the progress achieved relative to the amination of alkylene oxides, there remains the need for a process and a catalyst which displays both an improved activity and selectivity in the amination reactions, in particular towards the mono- and dialkylated amine products, and yet more towards the monoalkylated amine products. In particular, there remains a need for a process and a catalyst, wherein the conversion of the alkylene oxide educts is practically complete, and wherein the production of the unwanted trialkylated amine products may be reduced to an absolute minimum, if not practically eliminated from the product spectrum.

Pouria, R. et al. describes a process for the catalytic cracking of propane on La-ZSM-5, wherein lanthanum is loaded onto HZSM-5 by wet impregnation and subsequent drying and calcining of the loaded zeolite.

### DETAILED DESCRIPTION

It was therefore the object of the present invention to provide a process for the amination of alkylene oxides, and in particular of ethylene oxide with ammonia, with an improved efficiency relative to the conversion of ethylene oxide, and which furthermore displays a high selectivity towards monoethanol amine, and a low selectivity towards triethanol amine. Said object is achieved by the inventive process. Thus, it has surprisingly been found that by specifically using a zeolitic catalyst material having a framework-type structure selected from the group consisting of MFI and/or MEL, including MEL/MFI intergrowths, and incorporating a rare earth metal into the zeolitic material, wherein the rare earth metal is not introduced into the zeolitic material via ion exchange but rather by introducing the rare earth metal as a metal salt and converting said salt to the rare earth metal oxide by calcination or a similar treatment, a highly improved process for the amination of ethylene oxide may be obtained displaying superior results both with regard to the activity as well as with regard to the selectivity of the amination reaction. In particular it has quite unexpectedly been found that in the amination of ethylene oxide, the selectivity of the reaction toward monoethanolamine may be substantially increased, wherein at the same time practically no triethanolamine side product is produced when employing the inventive process.

Therefore, the present invention relates to a process for the conversion of ethylene oxide to 2-aminoethanol and/or Di(2-hydroxyethyl)amine comprising (i) providing a catalyst comprising a zeolitic material comprising YO₂ and X₂O₃ in its framework structure, wherein Y is a tetravalent element and X is a trivalent element, wherein the zeolitic material has a framework-type structure selected from the group consisting of MFI and/or MEL, including MEL/MFI intergrowths, and wherein the zeolitic material contains one or more rare earth elements; (ii) providing a mixture in the liquid phase comprising ethylene oxide and ammonia; (iii) contacting the catalyst provided in (i) with the mixture in the liquid phase provided in (ii) for converting ethylene oxide to 2-aminoethanol and/or Di(2-hydroxyethyl)amine, wherein the catalyst provided in (i) is obtained and/or obtainable by a process comprising loading one or more salts of the one or more rare earth elements into the pores of the porous structure of the zeolitic material and optionally on the surface of the zeolitic material.

As disclosed above, the catalyst provided in (i) comprises a zeolitic material having a framework-type structure selected from the group consisting of MFI and/or MEL, including MEL/MFI intergrowths. It is preferred that the zeolitic material has an MFI or an MEL/MFI intergrowth framework-type structure, wherein more preferably the zeolitic material has an MFI framework-type structure.

In the case where the zeolitic material has an MFI framework-type structure, no particular restriction applies as regards the zeolitic material itself. It is preferred that the zeolitic material comprises one or more zeolites selected from the group consisting of Silicalite, ZSM-5, [Fe—Si—O]-MFI, Monoclinic H-ZSM-5, [Ga—Si—O]-MFI, [As—Si—O]-MFI, AMS-1B, AZ-1, Bor-C, Encilite, Boralite C, FZ-1, LZ-105, Mutinaite, NU-4, NU-5, TS-1, TSZ, TSZ-III, TZ-01, USC-4, USI-108, ZBH, ZKQ-1B, ZMQ-TB, organic-free ZSM-5, and mixtures of two or more thereof, more preferably from the group consisting of ZSM-5, AMS-1B, AZ-1, FZ-1, LZ-105, NU-4, NU-5, TSZ, TSZ-III, TZ-01, USC-4, USI-108, ZBH, ZKQ-1B, ZMQ-TB, and mixtures of two or more thereof, wherein more preferably the zeolitic material comprises ZSM-5, wherein more preferably the zeolitic material is ZSM-5.

As disclosed above, the catalyst provided in (i) comprises a zeolitic material having a framework-type structure selected from the group consisting of MFI and/or MEL, including MEL/MFI intergrowths. It is preferred that the zeolitic material has an MEL/MFI intergrowth framework-type structure. In the case where the zeolitic material has an MEL/MFI intergrowth framework-type structure, no particular restriction applies as regards the zeolitic material itself. It is preferred that the zeolitic material comprises Bor-D and/or ZBM-10, more preferably ZBM-10, wherein more preferably the zeolitic material is ZBM-10.

As disclosed above, the catalyst provided in (i) comprises a zeolitic material having a framework-type structure selected from the group consisting of MFI and/or MEL, including MEL/MFI intergrowths. It is preferred that the zeolitic material has an MEL framework-type structure. In the case where the zeolitic material has an MEL framework-type structure, no particular restriction applies as regards the zeolitic material itself. It is preferred that the zeolitic material comprises one or more zeolites selected from the group consisting of Silicalite 2, ZSM-11, Boralite D, TS-2, SSZ-46, |DEOTA|[Si—B—O]-MEL, and mixtures of two or more thereof, more preferably from the group consisting of Silicalite 2, ZSM-11, TS-2, SSZ-46, and mixtures of two or more thereof, wherein more preferably the zeolitic material comprises ZSM-11 and/or SSZ-46, preferably ZSM-11, wherein more preferably the zeolitic material is ZSM-11 and/or SSZ-46, preferably ZSM-11.

As regards the YO₂:X₂O₃ molar ratio of the framework of the zeolitic material, no particular restriction applies. It is preferred that the framework of the zeolitic material displays a YO₂:X₂O₃ molar ratio in the range of from 5 to 300, more preferably from 10 to 200, more preferably from 15 to 150, more preferably from 20 to 120, more preferably from 25 to 100, more preferably from 30 to 80, more preferably from 35 to 70, more preferably from 40 to 60, and more preferably from 45 to 55.

As regards the element Y in the framework of the zeolitic material, no particular restriction applies provided that Y is a tetravalent element. Preferably, Y is selected from the group consisting of Si, Sn, Ti, Zr, Ge, and mixtures of two or more thereof, more preferably from the group consisting of Si, Ge, and mixtures thereof, more preferably Y being Si.

As regards the element X in the framework of the zeolitic material, no particular restriction applies provided that X is a trivalent element. Preferably, X is selected from the group consisting of Al, B, In, Ga, and mixtures of two or more thereof, X more preferably being Al and/or B, and more preferably being Al.

Therefore, it is particularly preferred that Y is selected from the group consisting of Si, Ge, and combinations thereof, that X is selected from the group consisting of Al, Ga, and combinations thereof, and that the framework of the zeolitic material displays a YO₂:X₂O₃ molar ratio in the range of from 5 to 300, more preferably from 10 to 200, more preferably from 15 to 150, more preferably from 20 to 120, more preferably from 25 to 100, more preferably from 30 to 80, more preferably from 35 to 70, more preferably from 40 to 60, and more preferably from 45 to 55.

As disclosed above, the zeolitic material of the catalyst provided in (i) contains one or more rare earth elements. As regards the one or more rare earth elements contained in the zeolitic material, no particular restriction applies. It is preferred that the one or more rare earth elements are selected from the group consisting of Ce, Dy, Er, Eu, Gd, Ho, La, Lu, Nd, Pr, Pm, Sm, Sc, Tb, Tm, Yb, and Y, more preferably from the group consisting of Ce, La, Nd, Pr, and Y, more preferably from the group consisting of Ce, La, and Y, wherein more preferably the one or more rare earth elements are La and/or Ce, preferably La.

As regards the amount of the one or more rare earth elements contained in the zeolitic material, no particular restriction applies. Preferably, the zeolitic material contains 1 to 15 weight-% of the one or more rare earth elements, calculated as the element and based on 100 weight-% of YO₂ contained in the framework structure of the zeolitic material, more preferably from 3 to 14 wt.-%, more preferably from 5 to 13 weight-%, more preferably from 5 to 11 weight-%, more preferably from 7 to 12.5 weight-%, more preferably from 9 to 12 weight-%, more preferably from 10 to 11.5 weight-%, and more preferably from 10.5 to 11 weight-%.

As regards the RE:X₂O₃ molar ratio of the one or more rare earth elements to X₂O₃ contained in the framework structure of the zeolitic material, no particular restriction applies. Preferably, the RE:X₂O₃ molar ratio of the one or more rare earth elements to X₂O₃ contained in the framework structure of the zeolitic material is in the range of from 0.1 to 6, more preferably from 0.3 to 5, more preferably from 0.5 to 4.5, more preferably from 0.8 to 4, more preferably from 1 to 3.8, more preferably from 1.2 to 3.6, more preferably from 1.4 to 3.4, more preferably from 1.5 to 3.2, more preferably from 1.6 to 3, more preferably from 1.8 to 2.8, more preferably from 2 to 2.6, and more preferably from 2.2 to 2.4.

As regards the zeolitic material of the catalyst provided in (i), no particular restriction applies in view of further elements contained therein. It is preferred that the zeolitic material contains substantially no Na, preferably substantially no Na or K, more preferably substantially no alkali metal, and more preferably substantially no alkali metal or alkaline earth metals. Within the meaning of the present invention, “substantially” as employed in the present invention with respect to the amount of Na, K, alkali metals or alkaline earth metals contained in the zeolitic material indicates an amount of 0.1 wt.-% or less of Na, K, alkali metals or alkaline earth metals calculated as the element and based on 100 wt.-% of YO₂ contained in the framework structure of the zeolitic material, preferably 0.05 wt.-% or less, more preferably 0.001 wt.-% or less, more preferably 0.0005 wt.-% or less, and even more preferably 0.0001 wt.-% or less thereof.

As regards the chemical properties of the catalyst provided in (i) comprising a zeolitic material, no particular restriction applies. Preferably, the catalyst provided in (i) and contacted with the mixture in the liquid phase in (iii) displays a Lewis acidity in the range of from 50 to 120, preferably from 60 to 110, more preferably from 65 to 108, more preferably from 70 to 106, more preferably from 72 to 104, more preferably from 74 to 102, more preferably from 76 to 100, more preferably from 78 to 98, more preferably from 80 to 96, more preferably from 82 to 94, more preferably from 84 to 92, more preferably from 86 to 90, and more preferably from 87 to 88. According to the present invention, the Lewis acidity is determined according to the procedure described in the experimental section of the present application.

As disclosed above, no particular restriction applies as regards the chemical properties of the catalyst provided in (i) comprising a zeolitic material. It is preferred that the catalyst provided in (i) and contacted with the mixture in the liquid phase in (iii) displays a Bronsted acidity in the range of from 2 to 35, more preferably from 4 to 30, more preferably from 6 to 28, more preferably from 8 to 26, more preferably from 10 to 24, more preferably from 12 to 22, more preferably from 14 to 20, and more preferably from 16 to 18. According to the present invention, the Bronsted acidity is determined according to the procedure described in the experimental section of the present application.

Further, no restriction applies as regards the ratio L:B of the Lewis acidity (L) to the Bronsted acidity (B) of the catalyst provided in (i) and contacted with the mixture in the liquid phase in (iii). Preferably, the ratio L:B of the Lewis acidity (L) to the Bronsted acidity (B) of the catalyst provided in (i) and contacted with the mixture in the liquid phase in (iii) is in the range of from 2 to 15, preferably from 2.5 to 12, more preferably from 3 to 10, more preferably from 3.5 to 9, more preferably from 4 to 8, more preferably from 4.5 to 7, more preferably from 5 to 6, and more preferably from 5 to 5.5.

Therefore, it is particularly preferred that the catalyst provided in (i) and contacted with the mixture in the liquid phase in (iii) displays a Lewis acidity in the range of from 50 to 120, preferably from 60 to 110, more preferably from 65 to 108, more preferably from 70 to 106, more preferably from 72 to 104, more preferably from 74 to 102, more preferably from 76 to 100, more preferably from 78 to 98, more preferably from 80 to 96, more preferably from 82 to 94, more preferably from 84 to 92, more preferably from 86 to 90, and more preferably from 87 to 88, that the catalyst provided in (i) and contacted with the mixture in the liquid phase in (iii) displays a Bronsted acidity in the range of from 2 to 35, more preferably from 4 to 30, more preferably from 6 to 28, more preferably from 8 to 26, more preferably from 10 to 24, more preferably from 12 to 22, more preferably from 14 to 20, and more preferably from 16 to 18, and that the ratio L:B of the Lewis acidity (L) to the Bronsted acidity (B) of the catalyst provided in (i) and contacted with the mixture in the liquid phase in (iii) is in the range of from 2 to 15, preferably from 2.5 to 12, more preferably from 3 to 10, more preferably from 3.5 to 9, more preferably from 4 to 8, more preferably from 4.5 to 7, more preferably from 5 to 6, and more preferably from 5 to 5.5.

As regards the acid sites of the catalyst provided in (i) and contacted with the mixture in the liquid phase in (iii), no particular restriction applies. Preferably, the catalyst provided in (i) and contacted with the mixture in the liquid phase in (iii) displays a total amount of acid sites as determined by NH₃-TPD in the range of from 0.1 to 2 mmol/g, preferably from 0.3 to 1.5 mmol/g, more preferably from 0.4 to 1.2 mmol/g, more preferably from 0.5 to 1 mmol/g, more preferably from 0.55 to 0.9 mmol/g, more preferably from 0.58 to 0.8 mmol/g, more preferably from 0.6 to 0.75 mmol/g, more preferably from 0.63 to 0.72 mmol/g, more preferably from 0.65 to 0.7 mmol/g, and more preferably from 0.67 to 0.68 mmol/g.

Further, as regards the weak acid sites of the catalyst provided in (i) and contacted with the mixture in the liquid phase in (iii), again no particular restriction applies. Preferably, the catalyst provided in (i) and contacted with the mixture in the liquid phase in (iii) displays an amount of weak acid sites as determined by NH₃-TPD in the range of from 0.1 to 0.9 mmol/g, more preferably from 0.2 to 0.7 mmol/g, more preferably from 0.3 to 0.6 mmol/g, more preferably from 0.35 to 0.55 mmol/g, more preferably from 0.4 to 0.5 mmol/g, more preferably from 0.43 to 0.48 mmol/g, and more preferably from 0.45 to 0.46 mmol/g.

Further, as regards the medium acid sites of the catalyst provided in (i) and contacted with the mixture in the liquid phase in (iii), again no particular restriction applies. Preferably, the catalyst provided in (i) and contacted with the mixture in the liquid phase in (iii) displays an amount of medium acid sites as determined by NH₃-TPD in the range of from 0.01 to 0.5 mmol/g, more preferably from 0.05 to 0.4 mmol/g, more preferably from 0.1 to 0.35 mmol/g, more preferably from 0.15 to 0.3 mmol/g, more preferably from 0.18 to 0.27 mmol/g, more preferably from 0.2 to 0.25 mmol/g, and more preferably from 0.22 to 0.23 mmol/g.

Further, as regards the strong acid sites of the catalyst provided in (i) and contacted with the mixture in the liquid phase in (iii), again no particular restriction applies. Preferably, the catalyst provided in (i) and contacted with the mixture in the liquid phase in (iii) displays an amount of strong acid sites as determined by NH₃-TPD of 0.05 mmol/g or less, more preferably of 0.01 mmol/g or less, more preferably of 0.005 mmol/g or less, more preferably of 0.001 mmol/g or less, more preferably of 0.0005 mmol/g or less, more preferably of 0.0001 mmol/g or less, more preferably of 0.00005 mmol/g or less, and more preferably of 0.00001 mmol/g or less.

Further, as regards the molar ratio of weak acid sites to medium acid sites as respectively determined by NH₃-TPD of the catalyst provided in (i) and contacted with the mixture in the liquid phase in (iii), no particular restriction applies. It is preferred that the molar ratio of weak acid sites to medium acid sites as respectively determined by NH₃-TPD of the catalyst provided in (i) and contacted with the mixture in the liquid phase in (iii) is in the range of from 0.1 to 5, more preferably from 0.5 to 3.5, more preferably from 1 to 3, more preferably from 1.2 to 2.8, more preferably from 1.4 to 2.6, more preferably from 1.6 to 2.4, more preferably from 1.8 to 2.2, and more preferably from 2 to 2.1.

As regards the physical properties of the catalyst provided in (i) comprising a zeolitic material, no particular restriction applies. It is preferred that the BET surface area of the catalyst provided in (i) and contacted with the mixture in the liquid phase in (iii) as determined according to ISO 9277:2010 is in the range of from 100 to 600 m²/g, more preferably from 150 to 500 m²/g, more preferably from 175 to 450 m²/g, more preferably from 200 to 400 m²/g, more preferably from 225 to 350 m²/g, more preferably from 250 to 300 m²/g, more preferably from 275 to 290 m²/g, and more preferably from 280 to 285 m²/g.

As disclosed above, the catalyst provided in (i) is obtained and/or obtainable by a process comprising loading one or more salts of the one or more rare earth elements into the pores of the porous structure of the zeolitic material and optionally on the surface of the zeolitic material. As regards said process, no particular restriction applies provided that one or more salts of the one or more rare earth elements are loaded into the pores of the porous structure of the zeolitic material and optionally on the surface of the zeolitic material. According to a first alternative which is referred to a wet impregnation, it is preferred that the loading of the one or more salts of the one or more rare earth elements into the pores of the porous structure of the zeolitic material and optionally on the surface of the zeolitic material comprises

(a) impregnating the porous structure of the zeolitic material with a solution of the one or more salts of the one or more rare earth elements;
(b) optionally drying the impregnated zeolitic material obtained in (b);
(c) calcining the zeolitic material obtained in (a) or (b).

Therefore, the present invention relates to a process for the conversion of ethylene oxide to 2-aminoethanol and/or Di(2-hydroxyethyl)amine comprising (i) providing a catalyst comprising a zeolitic material comprising YO₂ and X₂O₃ in its framework structure, wherein Y is a tetravalent element and X is a trivalent element, wherein the zeolitic material has a framework-type structure selected from the group consisting of MFI and/or MEL, including MEL/MFI intergrowths, and wherein the zeolitic material contains one or more rare earth elements; (ii) providing a mixture in the liquid phase comprising ethylene oxide and ammonia; (iii) contacting the catalyst provided in (i) with the mixture in the liquid phase provided in (ii) for converting ethylene oxide to 2-aminoethanol and/or Di(2-hydroxyethyl)amine, wherein the catalyst provided in (i) is obtained and/or obtainable by a process comprising loading one or more salts of the one or more rare earth elements into the pores of the porous structure of the zeolitic material and optionally on the surface of the zeolitic material, wherein the latter process comprises (a) impregnating the porous structure of the zeolitic material with a solution of the one or more salts of the one or more rare earth elements; (b) optionally drying the impregnated zeolitic material obtained in (b); (c) calcining the zeolitic material obtained in (a) or (b).

In the case where the catalyst provided in (i) is obtained and/or obtainable by a process comprising the steps (a), (b), and (c) as disclosed above, no particular restriction applies as regards the nature of the solution. It is preferred that the solution is an aqueous solution, wherein more preferably the solution consists of the one or more salts of the one or more rare earth elements dissolved in distilled water.

Further, in the case where the catalyst provided in (i) is obtained and/or obtainable by a process comprising the steps (a), (b), and (c) as disclosed above, no particular restriction applies as regards the ratio of the volume of the solution employed in (a) to the total pore volume of the zeolitic material prior to impregnation with the solution. It is preferred that the volume of the solution employed in (a) is equal to 500% or less of the total pore volume of the zeolitic material prior to impregnation with the solution, wherein more preferably the volume of the solution employed in (a) is equal to 50 to 350% of the total pore volume of the zeolitic material prior to impregnation with the solution, more preferably to 100 to 300%, more preferably to 150 to 270%, more preferably to 180 to 250%, more preferably to 200 to 230%, and more preferably to 210 to 220%. According to the present invention, the total pore volume is determined by nitrogen adsorption from the BJH method, preferably according to DIN 66134.

Further, in the case where the catalyst provided in (i) is obtained and/or obtainable by a process comprising the steps (a), (b), and (c) as disclosed above, no particular restriction applies as regards the temperature at which (a) is conducted. It is preferred that (a) is conducted at a temperature in the range of from 5 to 40° C., preferably from 10 to 35° C., more preferably from 15 to 30° C., and more preferably from 20 to 25° C.

Further, in the case where the catalyst provided in (i) is obtained and/or obtainable by a process comprising the steps (a), (b), and (c) as disclosed above, no particular restriction applies as regards the nature of the one or more salts. It is preferred that the one or more salts are selected from the group consisting of halides, more preferably chloride and/or bromide, more preferably chloride, hydroxide, sulfate, nitrate, phosphate, acetate, and mixtures of two or more thereof, more preferably from the group consisting of chloride, acetate, nitrate, and mixtures of two or more thereof, wherein more preferably the one or more salts are nitrates.

As disclosed above, the catalyst provided in (i) is obtained and/or obtainable by a process comprising loading one or more salts of the one or more rare earth elements into the pores of the porous structure of the zeolitic material and optionally on the surface of the zeolitic material. As regards said process, no particular restriction applies provided that one or more salts of the one or more rare earth elements are loaded into the pores of the porous structure of the zeolitic material and optionally on the surface of the zeolitic material. According to a second alternative which is referred to a solid state impregnation, it is preferred that the loading of the one or more salts of the one or more rare earth elements into the pores of the porous structure of the zeolitic material and optionally on the surface of the zeolitic material comprises

(a′) preparing a mixture of the one or more salts of the one or more rare earth elements and the zeolitic material;
(b′) optionally milling the mixture obtained in (a′);
(c′) calcining the zeolitic material obtained in (a′) or (b′).

Therefore, the present invention relates to a process for the conversion of ethylene oxide to 2-aminoethanol and/or Di(2-hydroxyethyl)amine comprising (i) providing a catalyst comprising a zeolitic material comprising YO₂ and X₂O₃ in its framework structure, wherein Y is a tetravalent element and X is a trivalent element, wherein the zeolitic material has a framework-type structure selected from the group consisting of MFI and/or MEL, including MEL/MFI intergrowths, and wherein the zeolitic material contains one or more rare earth elements; (ii) providing a mixture in the liquid phase comprising ethylene oxide and ammonia; (iii) contacting the catalyst provided in (i) with the mixture in the liquid phase provided in (ii) for converting ethylene oxide to 2-aminoethanol and/or Di(2-hydroxyethyl)amine, wherein the catalyst provided in (i) is obtained and/or obtainable by a process comprising loading one or more salts of the one or more rare earth elements into the pores of the porous structure of the zeolitic material and optionally on the surface of the zeolitic material, wherein the latter process comprises (a′) preparing a mixture of the one or more salts of the one or more rare earth elements and the zeolitic material; (b′) optionally milling the mixture obtained in (a′); (c′) calcining the zeolitic material obtained in (a′) or (b′).

In the case where the catalyst provided in (i) is obtained and/or obtainable by a process comprising the steps (a′), (b′), and (c′) as disclosed above for the solid state impregnation, no particular restriction applies as regards the nature of the one or more salts. It is preferred that the one or more salts are selected from the group consisting of halides, preferably chloride and/or bromide, more preferably chloride, hydroxide, sulfate, nitrate, phosphate, acetate, and mixtures of two or more thereof, more preferably from the group consisting of chloride, acetate, nitrate, and mixtures of two or more thereof, wherein more preferably the one or more salts are nitrates.

In the case where the catalyst provided in (i) is obtained and/or obtainable by a process comprising the steps (a), (b), and (c) as disclosed above for the wet impregnation or comprising the steps (a′), (b′), and (c′) as disclosed above for the solid state impregnation, no particular restriction applies as regards the conditions, in particular as regards the temperature, under which calcining in (c) or (c′) is conducted. It is preferred that the calcining in (c) or (c′) is conducted at a temperature in the range of from 300 to 900° C., more preferably of from 350 to 700° C., more preferably of from 400 to 600° C., and more preferably of from 450 to 550° C.

Further, in the case where the catalyst provided in (i) is obtained and/or obtainable by a process comprising the steps (a), (b), and (c) as disclosed above for the wet impregnation or comprising the steps (a′), (b′), and (c′) as disclosed above for the solid state impregnation, no particular restriction applies as regards the conditions, in particular the composition of the gas mixture, under which calcining in (c) or (c′) is conducted. It is preferred that calcining in (c) or (c′) is conducted in air.

Further, in the case where the catalyst provided in (i) is obtained and/or obtainable by a process comprising the steps (a), (b), and (c) as disclosed above for the wet impregnation or comprising the steps (a′), (b′), and (c′) as disclosed above for the solid state impregnation, no particular restriction applies as regards the condition of the zeolitic material prior to the loading of the one or more salts of the one or more rare earth elements into the pores of the porous structure of the zeolitic material and optionally on the surface of the zeolitic material in (a) or (a′). It is preferred that prior to the loading of the one or more salts of the one or more rare earth elements into the pores of the porous structure of the zeolitic material and optionally on the surface of the zeolitic material in (a) or (a′), the zeolitic material is in the H-form and contains protons as extra-framework ions. More preferably the zeolitic material is in the H-form and contains protons as extra-framework ions, wherein 0.1 weight-% or less of the extra-framework ions are metal cations, calculated as the element and based on 100 weight-% of YO₂ contained in the zeolitic material, more preferably 0.05 weight-% or less, more preferably 0.001 weight-% or less, more preferably 0.0005 weight-% or less, and more preferably 0.0001 weight-% or less.

As disclosed above, the present invention relates to a process for the conversion of ethylene oxide to 2-aminoethanol and/or Di(2-hydroxyethyl)amine comprising (i) providing a catalyst comprising a zeolitic material comprising YO₂ and X₂O₃ in its framework structure, wherein Y is a tetravalent element and X is a trivalent element, wherein the zeolitic material has a framework-type structure selected from the group consisting of MFI and/or MEL, including MEL/MFI intergrowths, and wherein the zeolitic material contains one or more rare earth elements; (ii) providing a mixture in the liquid phase comprising ethylene oxide and ammonia; (iii) contacting the catalyst provided in (i) with the mixture in the liquid phase provided in (ii) for converting ethylene oxide to 2-aminoethanol and/or Di(2-hydroxyethyl)amine, wherein the catalyst provided in (i) is obtained and/or obtainable by a process comprising loading one or more salts of the one or more rare earth elements into the pores of the porous structure of the zeolitic material and optionally on the surface of the zeolitic material.

As regards the catalyst provided in (i) and contacted with the mixture in the liquid phase in (iii), no particular restriction applies on how the one or more salts of the one or more rare earth elements are loaded into the pores of the porous structure of the zeolitic material and optionally on the surface of the zeolitic material. It is preferred that the catalyst provided in (i) and contacted with the mixture in the liquid phase in (iii) is obtained and/or obtainable by a process which does not comprise a step of ion exchanging the one or more rare earth elements into the zeolitic material.

As regards the temperature at which the contacting in (iii) is effected, no particular restriction applies. It is preferred that the contacting in (iii) is effected at a temperature in the range of from 40 to 180° C., more preferably from 50 to 150° C., more preferably from 55 to 130° C., more preferably from 60 to 120° C., more preferably from 65 to 115° C., more preferably from 70 to 110° C., more preferably from 75 to 105° C., more preferably from 80 to 100° C., and more preferably from 85 to 95° C.

As regards the pressure at which the contacting in (iii) is effected, no particular restriction applies. It is preferred that the contacting in (iii) is effected at a pressure in the range of from 50 to 250 bar, more preferably of from 80 to 200 bar, more preferably of from 100 to 180 bar, more preferably of from 110 to 170 bar, more preferably of from 120 to 150 bar, more preferably of from 125 to 145 bar, and more preferably of from 130 to 140 bar.

Therefore, it is particularly preferred that the contacting in (iii) is effected at a temperature in the range of from 40 to 180° C., more preferably from 50 to 150° C., more preferably from 55 to 130° C., more preferably from 60 to 120° C., more preferably from 65 to 115° C., more preferably from 70 to 110° C., more preferably from 75 to 105° C., more preferably from 80 to 100° C., and more preferably from 85 to 95° C., and at a pressure in the range of from 50 to 250 bar, more preferably of from 80 to 200 bar, more preferably of from 100 to 180 bar, more preferably of from 110 to 170 bar, more preferably of from 120 to 150 bar, more preferably of from 125 to 145 bar, and more preferably of from 130 to 140 bar.

As regards the ammonia:ethylene oxide molar ratio in the mixture in the liquid phase provided in (ii) and contacted with the catalyst in (iii), no particular restriction applies. Preferably, the ammonia:ethylene oxide molar ratio in the mixture in the liquid phase provided in (ii) and contacted with the catalyst in (iii) is in the range of from 6 to 50, more preferably from 8 to 45, more preferably from 10 to 40, more preferably from 12 to 35, more preferably from 14 to 30, more preferably from 16 to 25, more preferably from 18 to 23, and more preferably from 20 to 21.

As regards the weight ratio H₂O:NH₃ of water to ammonia in the mixture in the liquid phase provided in (ii) and contacted with the catalyst in (iii), no particular restriction applies. Preferably, the weight ratio H₂O:NH₃ of water to ammonia in the mixture in the liquid phase provided in (ii) and contacted with the catalyst in (iii) is in the range of from 0 to 30, more preferably of from 0 to 20, more preferably of from 0 to 15, more preferably of from 0 to 10, more preferably of from 0 to 7, more preferably of from 0 to 5, more preferably of from 0 to 3, more preferably of from 0 to 2, and more preferably of from 0 to 1.

As regards the amounts of ammonia and ethylene oxide in the mixture in the liquid phase provided in (ii) and contacted with the catalyst in (iii), no particular restriction applies. Preferably, the mixture in the liquid phase provided in (ii) and contacted with the catalyst in (iii) consists of 50 weight-% or more of ammonia and ethylene oxide, more preferably 60 weight-% or more, more preferably 70 weight-% or more, more preferably 80 weight-% or more, more preferably 90 weight-% or more, more preferably 95 weight-% or more, more preferably 99 weight-% or more, and more preferably 99.9 weight-% or more.

Therefore, it is particularly preferred that the ammonia:ethylene oxide molar ratio in the mixture in the liquid phase provided in (ii) and contacted with the catalyst in (iii) is in the range of from 6 to 50, preferably from 8 to 45, more preferably from 10 to 40, more preferably from 12 to 35, more preferably from 14 to 30, more preferably from 16 to 25, more preferably from 18 to 23, and more preferably from 20 to 21, and that the weight ratio H₂O:NH₃ of water to ammonia in the mixture in the liquid phase provided in (ii) and contacted with the catalyst in (iii) is in the range of from 0 to 30, preferably of from 0 to 20, more preferably of from 0 to 15, more preferably of from 0 to 10, more preferably of from 0 to 7, more preferably of from 0 to 5, more preferably of from 0 to 3, more preferably of from 0 to 2, and more preferably of from 0 to 1, and that the mixture in the liquid phase provided in (ii) and contacted with the catalyst in (iii) consists of 50 weight-% or more of ammonia and ethylene oxide, preferably 60 weight-% or more, more preferably 70 weight-% or more, more preferably 80 weight-% or more, more preferably 90 weight-% or more, more preferably 95 weight-% or more, more preferably 99 weight-% or more, and more preferably 99.9 weight-% or more.

The present invention is further illustrated by the following embodiments and combinations of embodiments as indicated by the respective dependencies and back-references. In particular, it is noted that in each instance where a combination of embodiments is mentioned as a range, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3, and 4”. Thus, the present invention includes the following embodiments, wherein these include the specific combinations of embodiments as indicated by the respective interdependencies defined therein:
1. A process for the conversion of ethylene oxide to 2-aminoethanol and/or Di(2-hydroxyethyl)amine comprising
   (i) providing a catalyst comprising a zeolitic material comprising YO₂ and X₂O₃ in its framework structure, wherein Y is a tetravalent element and X is a trivalent element, wherein the zeolitic material has a framework-type structure selected from the group consisting of MFI and/or MEL, including MEL/MFI intergrowths, and wherein the zeolitic material contains one or more rare earth elements;
   (ii) providing a mixture in the liquid phase comprising ethylene oxide and ammonia;
   (iii) contacting the catalyst provided in (i) with the mixture in the liquid phase provided in (ii) for converting ethylene oxide to 2-aminoethanol and/or Di(2-hydroxyethyl)amine, wherein the catalyst provided in (i) is obtained and/or obtainable by a process comprising loading one or more salts of the one or more rare earth elements into the pores of the porous structure of the zeolitic material and optionally on the surface of the zeolitic material.
2. The process of embodiment 1, wherein the zeolitic material has an MFI or an MEL/MFI intergrowth framework-type structure, wherein more preferably the zeolitic material has an MFI framework-type structure.
3. The process of embodiment 1 or 2, wherein the catalyst provided in (i) comprises a zeolitic material having an MFI framework-type structure, wherein the zeolitic material preferably comprises one or more zeolites selected from the group consisting of Silicalite, ZSM-5, [Fe—Si—O]-MFI, Monoclinic H-ZSM-5, [Ga—Si—O]-MFI, [As—Si—O]-MFI, AMS-1B, AZ-1, Bor-C, Encilite, Boralite C, FZ-1, LZ-105, Mutinaite, NU-4, NU-5, TS-1, TSZ, TSZ-III, TZ-01, USC-4, USI-108, ZBH, ZKQ-1B, ZMQ-TB, organic-free ZSM-5, and mixtures of two or more thereof, more preferably from the group consisting of ZSM-5, AMS-1B, AZ-1, FZ-1, LZ-105, NU-4, NU-5, TSZ, TSZ-III, TZ-01, USC-4, USI-108, ZBH, ZKQ-1B, ZMQ-TB, and mixtures of two or more thereof, wherein more preferably the zeolitic material comprises ZSM-5, wherein more preferably the zeolitic material is ZSM-5.
4. The process of any of embodiments 1 to 3, wherein the catalyst provided in (i) comprises a zeolitic material having an MEL/MFI intergrowth framework-type structure, wherein the zeolitic material preferably comprises Bor-D and/or ZBM-10, preferably ZBM-10, wherein more preferably the zeolitic material is ZBM-10.
5. The process of any of embodiments 1 to 4, wherein the catalyst provided in (i) comprises a zeolitic material having an MEL framework-type structure, wherein the zeolitic material preferably comprises one or more zeolites selected from the group consisting of Silicalite 2, ZSM-11, Boralite D, TS-2, SSZ-46, IDEOTAI[Si—B—O]-MEL, and mixtures of two or more thereof, more preferably from the group consisting of Silicalite 2, ZSM-11, TS-2, SSZ-46, and mixtures of two or more thereof, wherein more preferably the zeolitic material comprises ZSM-11 and/or SSZ-46, preferably ZSM-11, wherein more preferably the zeolitic material is ZSM-11 and/or SSZ-46, preferably ZSM-11.
6. The process of any of embodiments 1 to 5, wherein the framework of the zeolitic material displays a YO₂:X₂O₃ molar ratio in the range of from 5 to 300, preferably from 10 to 200, more preferably from 15 to 150, more preferably from 20 to 120, more preferably from 25 to 100, more preferably from 30 to 80, more preferably from 35 to 70, more preferably from 40 to 60, and more preferably from 45 to 55.
7. The process of any of embodiments 1 to 6, wherein Y is selected from the group consisting of Si, Sn, Ti, Zr, Ge, and mixtures of two or more thereof, Y preferably being Si.
8. The process of any of embodiments 1 to 7, wherein X is selected from the group consisting of Al, B, In, Ga, and mixtures of two or more thereof, X preferably being Al and/or B, and more preferably being Al.
9. The process of any of embodiments 1 to 8, wherein the one or more rare earth elements are selected from the group consisting of
   Ce, Dy, Er, Eu, Gd, Ho, La, Lu, Nd, Pr, Pm, Sm, Sc, Tb, Tm, Yb, and Y, preferably from the group consisting of Ce, La, Nd, Pr, and Y, more preferably from the group consisting of Ce, La, and Y, wherein more preferably the one or more rare earth elements are La and/or Ce, preferably La.
10. The process of any of embodiments 1 to 9, wherein the zeolitic material contains 1 to 15 wt.-% of the one or more rare earth elements, calculated as the element and based on 100 wt.-% of YO₂ contained in the framework structure of the zeolitic material, preferably from 3 to 14 wt.-%, more preferably from 5 to 13 wt.-%, more preferably from 5 to 11 wt. %, more preferably from 7 to 12.5 wt.-%, more preferably from 9 to 12 wt.-%, more preferably from 10 to 11.5 wt.-%, and more preferably from 10.5 to 11 wt.-%.
11. The process of any of embodiments 1 to 10, wherein the RE:X₂O₃ molar ratio of the one or more rare earth elements to X₂O₃ contained in the framework structure of the zeolitic material is in the range of from 0.1 to 6, preferably from 0.3 to 5, more preferably from 0.5 to 4.5, more preferably from 0.8 to 4, more preferably from 1 to 3.8, more preferably from 1.2 to 3.6, more preferably from 1.4 to 3.4, more preferably from 1.5 to 3.2, more preferably from 1.6 to 3, more preferably from 1.8 to 2.8, more preferably from 2 to 2.6, and more preferably from 2.2 to 2.4.
12. The process of any of embodiments 1 to 11, wherein the zeolitic material contains substantially no Na, preferably substantially no Na or K, more preferably substantially no alkali metal, and more preferably substantially no alkali metal or alkaline earth metals.
13. The process of any of embodiments 1 to 12, wherein the catalyst provided in (i) and contacted with the mixture in the liquid phase in (iii) displays a Lewis acidity in the range of from 50 to 120, preferably from 60 to 110, more preferably from 65 to 108, more preferably from 70 to 106, more preferably from 72 to 104, more preferably from 74 to 102, more preferably from 76 to 100, more preferably from 78 to 98, more preferably from 80 to 96, more preferably from 82 to 94, more preferably from 84 to 92, more preferably from 86 to 90, and more preferably from 87 to 88.
14. The process of any of embodiments 1 to 13, wherein the catalyst provided in (i) and contacted with the mixture in the liquid phase in (iii) displays a Bronsted acidity in the range of from 2 to 35, preferably from 4 to 30, more preferably from 6 to 28, more preferably from 8 to 26, more preferably from 10 to 24, more preferably from 12 to 22, more preferably from 14 to 20, and more preferably from 16 to 18.
15. The process of any of embodiments 1 to 14, wherein the ratio L:B of the Lewis acidity (L) to the Bronsted acidity (B) of the catalyst provided in (i) and contacted with the mixture in the liquid phase in (iii) is in the range of from 2 to 15, preferably from 2.5 to 12, more preferably from 3 to 10, more preferably from 3.5 to 9, more preferably from 4 to 8, more preferably from 4.5 to 7, more preferably from 5 to 6, and more preferably from 5 to 5.5.
16. The process of any of embodiments 1 to 15, wherein the catalyst provided in (i) and contacted with the mixture in the liquid phase in (iii) displays a total amount of acid sites as determined by NH₃-TPD in the range of from 0.1 to 2 mmol/g, preferably from 0.3 to 1.5 mmol/g, more preferably from 0.4 to 1.2 mmol/g, more preferably from 0.5 to 1 mmol/g, more preferably from 0.55 to 0.9 mmol/g, more preferably from 0.58 to 0.8 mmol/g, more preferably from 0.6 to 0.75 mmol/g, more preferably from 0.63 to 0.72 mmol/g, more preferably from 0.65 to 0.7 mmol/g, and more preferably from 0.67 to 0.68 mmol/g.
17. The process of any of embodiments 1 to 16, wherein the catalyst provided in (i) and contacted with the mixture in the liquid phase in (iii) displays an amount of weak acid sites as determined by NH₃-TPD in the range of from 0.1 to 0.9 mmol/g, preferably from 0.2 to 0.7 mmol/g, more preferably from 0.3 to 0.6 mmol/g, more preferably from 0.35 to 0.55 mmol/g, more preferably from 0.4 to 0.5 mmol/g, more preferably from 0.43 to 0.48 mmol/g, and more preferably from 0.45 to 0.46 mmol/g.
18. The process of any of embodiments 1 to 17, wherein the catalyst provided in (i) and contacted with the mixture in the liquid phase in (iii) displays an amount of medium acid sites as determined by NH₃-TPD in the range of from 0.01 to 0.5 mmol/g, preferably from 0.05 to 0.4 mmol/g, more preferably from 0.1 to 0.35 mmol/g, more preferably from 0.15 to 0.3 mmol/g, more preferably from 0.18 to 0.27 mmol/g, more preferably from 0.2 to 0.25 mmol/g, and more preferably from 0.22 to 0.23 mmol/g.
19. The process of any of embodiments 1 to 18, wherein the catalyst provided in (i) and contacted with the mixture in the liquid phase in (iii) displays an amount of strong acid sites as determined by NH₃-TPD of 0.05 mmol/g or less, more preferably of 0.01 mmol/g or less, more preferably of 0.005 mmol/g or less, more preferably of 0.001 mmol/g or less, more preferably of 0.0005 mmol/g or less, more preferably of 0.0001 mmol/g or less, more preferably of 0.00005 mmol/g or less, and more preferably of 0.00001 mmol/g or less.
20. The process of any of embodiments 1 to 19, wherein the molar ratio of weak acid sites to medium acid sites as respectively determined by NH₃-TPD of the catalyst provided in (i) and contacted with the mixture in the liquid phase in (iii) is in the range of from 0.1 to 5, preferably from 0.5 to 3.5, more preferably from 1 to 3, more preferably from 1.2 to 2.8, more preferably from 1.4 to 2.6, more preferably from 1.6 to 2.4, more preferably from 1.8 to 2.2, and more preferably from 2 to 2.1.
21. The process of any of embodiments 1 to 20, wherein the BET surface area of the catalyst provided in (i) and contacted with the mixture in the liquid phase in (iii) as determined according to ISO 9277:2010 is in the range of from 100 to 600 m²/g, preferably from 150 to 500 m²/g, more preferably from 175 to 450 m²/g, more preferably from 200 to 400 m²/g, more preferably from 225 to 350 m²/g, more preferably from 250 to 300 m²/g, more preferably from 275 to 290 m²/g, and more preferably from 280 to 285 m²/g.
22. The process of any of embodiments 1 to 21, wherein the loading of the one or more salts of the one or more rare earth elements into the pores of the porous structure of the zeolitic material and optionally on the surface of the zeolitic material comprises
   (a) impregnating the porous structure of the zeolitic material with a solution of the one or more salts of the one or more rare earth elements;
   (b) optionally drying the impregnated zeolitic material obtained in (b);
   (c) calcining the zeolitic material obtained in (a) or (b).
23. The process of embodiment 22, wherein the solution is an aqueous solution, wherein preferably the solution consists of the one or more salts of the one or more rare earth elements dissolved in distilled water.
24. The process of embodiment 22 or 23, wherein the volume of the solution employed in (a) is equal to 500% or less of the total pore volume of the zeolitic material prior to impregnation with the solution, wherein preferably the volume of the solution employed in (a) is equal to 50 to 350% of the total pore volume of the zeolitic material prior to impregnation with the solution, more preferably to 100 to 300%, more preferably to 150 to 270%, more preferably to 180 to 250%, more preferably to 200 to 230%, and more preferably to 210 to 220%, wherein the total pore volume is determined by nitrogen adsorption from the BJH method, preferably according to DIN 66134.
25. The process of any of embodiments 22 to 24, wherein (a) is conducted at a temperature in the range of from 5 to 40° C., preferably from 10 to 35° C., more preferably from 15 to 30° C., and more preferably from 20 to 25° C.
26. The process of any of embodiments 22 to 25, wherein the one or more salts are selected from the group consisting of halides, preferably chloride and/or bromide, more preferably chloride, hydroxide, sulfate, nitrate, phosphate, acetate, and mixtures of two or more thereof, more preferably from the group consisting of chloride, acetate, nitrate, and mixtures of two or more thereof, wherein more preferably the one or more salts are nitrates.
27. The process of any of embodiments 1 to 21, wherein the loading of the one or more salts of the one or more rare earth elements into the pores of the porous structure of the zeolitic material and optionally on the surface of the zeolitic material comprises
   (a′) preparing a mixture of the one or more salts of the one or more rare earth elements and the zeolitic material;
   (b′) optionally milling the mixture obtained in (a′);
   (c′) calcining the zeolitic material obtained in (a′) or (b′).
28. The process of embodiment 27, wherein the one or more salts are selected from the group consisting of halides, preferably chloride and/or bromide, more preferably chloride, hydroxide, sulfate, nitrate, phosphate, acetate, and mixtures of two or more thereof, more preferably from the group consisting of chloride, acetate, nitrate, and mixtures of two or more thereof, wherein more preferably the one or more salts are nitrates.
29. The process of any of embodiments 22 to 28, wherein calcining in (c) or (c′) is conducted at a temperature in the range of from 300 to 900° C., preferably of from 350 to 700° C., more preferably of from 400 to 600° C., and more preferably of from 450 to 550° C.
30. The process of any of embodiments 22 to 29, wherein calcining in (c) or (c′) is conducted in air.
31. The process of any of embodiments 22 to 30, wherein prior to the loading of the one or more salts of the one or more rare earth elements into the pores of the porous structure of the zeolitic material and optionally on the surface of the zeolitic material in (a) or (a′), the zeolitic material is in the H-form and contains protons as extra-framework ions, wherein 0.1 wt.-% or less of the extra-framework ions are metal cations, calculated as the element and based on 100 wt.-% of YO₂ contained in the zeolitic material, preferably 0.05 wt.-% or less, more preferably 0.001 wt.-% or less, more preferably 0.0005 wt.-% or less, and more preferably 0.0001 wt.-% or less.
32. The process of any of embodiments 1 to 31, wherein the catalyst provided in (i) and contacted with the mixture in the liquid phase in (iii) is obtained and/or obtainable by a process which does not comprise a step of ion exchanging the one or more rare earth elements into the zeolitic material.
33. The process of any of embodiments 1 to 32, wherein the contacting in (iii) is effected at a temperature in the range of from 40 to 180° C., preferably from 50 to 150° C., more preferably from 55 to 130° C., more preferably from 60 to 120° C., more preferably from 65 to 115° C., more preferably from 70 to 110° C., more preferably from 75 to 105° C., more preferably from 80 to 100° C., and more preferably from 85 to 95° C.
34. The process of any of embodiments 1 to 33, wherein the contacting in (iii) is effected at a pressure in the range of from 50 to 250 bar, preferably of from 80 to 200 bar, more preferably of from 100 to 180 bar, more preferably of from 110 to 170 bar, more preferably of from 120 to 150 bar, more preferably of from 125 to 145 bar, and more preferably of from 130 to 140 bar.
35. The process of any of embodiments 1 to 34, wherein the ammonia:ethylene oxide molar ratio in the mixture in the liquid phase provided in (ii) and contacted with the catalyst in (iii) is in the range of from 6 to 50, preferably from 8 to 45, more preferably from 10 to 40, more preferably from 12 to 35, more preferably from 14 to 30, more preferably from 16 to 25, more preferably from 18 to 23, and more preferably from 20 to 21.
36. The process of any of embodiments 1 to 35, wherein the weight ratio H₂O:NH₃ of water to ammonia in the mixture in the liquid phase provided in (ii) and contacted with the catalyst in (iii) is in the range of from 0 to 30, preferably of from 0 to 20, more preferably of from 0 to 15, more preferably of from 0 to 10, more preferably of from 0 to 7, more preferably of from 0 to 5, more preferably of from 0 to 3, more preferably of from 0 to 2, and more preferably of from 0 to 1.
37. The process of any of embodiments 1 to 36, wherein the mixture in the liquid phase provided in (ii) and contacted with the catalyst in (iii) consists of 50 wt.-% or more of ammonia and ethylene oxide, preferably 60 wt.-% or more, more preferably 70 wt.-% or more, more preferably 80 wt.-% or more, more preferably 90 wt.-% or more, more preferably 95 wt.-% or more, more preferably 99 wt.-% or more, and more preferably 99.9 wt.-% or more.

### EXPERIMENTAL SECTION

Determination of the Bronsted and Lewis Acidities

In the examples, the Bronsted and Lewis acidities were determined using pyridine as the probe gas. The measurements were conducted using an IR-spectrometer Nicolet 6700 employing a HV-FTIR-cell. The samples were pressed to a pellet for placing in the HV-FTIR-cell for measurement. After being placed in the HV-FTIR-cell, the samples were then heated in air to 350° C. and held at that temperature for 1 h for removing water and any volatile substances from the sample. The apparatus was then placed under high-vacuum (10⁻⁵ mbar), and the cell let cool to 80° C., at which it was held for the entire duration of the measurement for avoiding the condensation of pyridine in the cell.

Pyridine was then dosed into the cell in successive steps (0.01, 0.1, 1, and 3 mbar) to ensure the controlled and complete exposition of the sample.

The irradiation spectrum of the activated sample at 80° C. and 10⁻⁵ mbar was used as the background for the absorption spectra for compensating the influence of matrix bands.

For the analysis, the spectrum at a pressure of 1 mbar was used, since the sample was in a stable equilibrium. For the quantification, the extinction spectrum was used, since this allowed for the cancellation of the matrix effects.

The integral extinction unit was determined as follows: the characteristic signals for the pyridine absorption were integrated and the area of the pellet was scaled with the thickness of the pellet.

Overview table: Assignment of the IR-bands of pyridine

| acid sites | pyridine species | bands (cm⁻¹) |
|---|---|---|
| L | Py | 1440-1455 |
| | | 1575 |
| | | 1620 |
| B | PyH⁺ | 1540-1550 |
| | | 1635-1640 |
| B + L | Py + PyH⁺ | 1490 |
| physical adsorbate | adsorbated Py | 1440 (overlay L) |
| | | 1580-1595 |

Py = pyridine;
PyH⁺ = pyridinium ion;
B = Bronsted center;
L = Lewis center

In the examples, the determination of the Lewis acid sites were determined using the band at 1450 cm⁻¹ and of the Bronsted acid sites using the band at 1545 cm⁻¹.

Temperature Programmed Desorption of Ammonia (NH₃-TPD)

The temperature-programmed desorption of ammonia (NH₃-TPD) was conducted in an automated chemisorption analysis unit (Micromeritics AutoChem II 2920) having a thermal conductivity detector. Continuous analysis of the desorbed species was accomplished using an online mass spectrometer (OmniStar QMG200 from Pfeiffer Vacuum). The sample (0.1 g) was introduced into a quartz tube and analysed using the program described below. The temperature was measured by means of a Ni/Cr/Ni thermocouple immediately above the sample in the quartz tube. For the analyses, He of purity 5.0 was used. Before any measurement, a blank sample was analysed for calibration.
1. Preparation: Commencement of recording; one measurement per second. Wait for 10 minutes at 25° C. and a He flow rate of 30 cm³/min (room temperature (about 25° C.) and 1 atm); heat up to 600° C. at a heating rate of 20 K/min; hold for 10 minutes. Cool down under a He flow (30 cm³/min) to 100° C. at a cooling rate of 20 K/min (furnace ramp temperature); Cool down under a He flow (30 cm³/min) to 100° C. at a cooling rate of 3 K/min (sample ramp temperature).
2. Saturation with NH₃: Commencement of recording; one measurement per second. Change the gas flow to a mixture of 10% NH₃ in He (75 cm³/min; 100° C. and 1 atm) at 100° C.; hold for 30 minutes.
3. Removal of the excess: Commencement of recording; one measurement per second. Change the gas flow to a He flow of 75 cm³/min (100° C. and 1 atm) at 100° C.; hold for 60 min.
4. NH₃-TPD: Commencement of recording; one measurement per second. Heat up under a He flow (flow rate: 30 cm³/min) to 600° C. at a heating rate of 10 K/min; hold for 30 minutes.
5. End of measurement.

Desorbed ammonia was measured by means of the online mass spectrometer, which demonstrates that the signal from the thermal conductivity detector was caused by desorbed ammonia. This involved utilizing the m/z=16 signal from ammonia in order to monitor the desorption of the ammonia. The amount of ammonia adsorbed (mmol/g of sample) was ascertained by means of the Micromeritics software through integration of the TPD signal with a horizontal baseline.

### Reference Example 1: Preparation of H-ZSM-5 (MFI-Type Framework Structure)

In a 2 m³ reactor 79.61 kg of de-ionised water is first introduced. To the water, 411.15 kg of an aqueous tetrapropylammonium hydroxide solution (TPAOH; 40 wt. %) was added under stirring (70 rpm). The suspension is let for stirring for another 10 min. 8.2 kg solid NaOH is added slowly in 2.5 kg portions under stirring and after each portion the system is allowed to mix for 5 minutes. Next, 29.25 kg aluminium triisopropoxide is added to the suspension and the system is stirred for another 1 h. At the end, 538.19 kg colloidal silica (Ludox AS-40) is added followed by additional 10 kg of de-ionized water. The synthesis mixture is stirred another 1 h at room temperature before the reactor is flushed with nitrogen gas and the pressure reduced to −900 mbar. Afterwards the reactor is heated to 170° C. in 11 h. The hydrothermal synthesis is run for 72 h at 170° C. under 70 rpm stirring. After crystallization the synthesis mixture is cooled down to 30° C. The suspension is transferred to a larger vessel where the pH of the suspension is adjusted to 7±0.5, by addition of a 10 wt. % aqueous nitric acid solution. The pH adjusted suspension is let for stirring for another 30 min at 70 rpm. The zeolite is separated by filtration and the filter cake is washed with de-ionised water until a conductivity of the wash water <200 μS. The filtercake is then dried at 120° C. for 96 h. The dried material was calcined to 550° C. in air for 6 h for obtaining a calcined ZSM-5 zeolite with a BET surface area of 390 m²/g, and displaying a crystallinity as determined by X-ray diffraction of 94%.

250 kg de-ionized water is added to a 400 L reactor and 25 kg ammonium nitrate is added under stirring (150 rpm). The suspension is heated to 80° C., followed by the addition of 25 kg of the calcined zeolite. The mixture is stirred further for 1 h at 80° C. Afterwards the reaction mixture is cooled down and filtered off using a filterpress and washed with water until a conductivity in the wash water <200 μS. The ion-exchange process is then repeated for obtaining an ammonium-exchanged ZSM-5. The filter cake obtained after the second ammonium ion-exchange process is dried for 10 h at 120° C. and calcined at 500° C. in air for 5 h (heating rate 2° C./min) for obtaining H-ZSM-5.

According to the elemental analysis the resulting product had the following contents determined per 100 g substance of <0.1 g carbon, 1.6 g aluminum, <0.01 g of sodium, and 43 g silicon.

The BET surface area was determined to be 408 m²/g.

### Reference Example 2: Preparation of ZBM-10 (MEL/MPI Intergrowth)

17.64 kg of distilled water were placed in a reaction vessel to which 10.14 kg of an aqueous hexamethylene diamine solution (70 wt.-% in distilled water), and subsequently 4.6 kg of fumed silica (Aerosil 200) were added under stirring. After mixing at 100 rpm for 5 min, the mixture was heated to 70° C., and the stirring speed then increased to 220 rpm. A solution of 1.01 kg Al₂(SO₄)₃*18 H₂O dissolved in 6.76 kg of distilled water was then stirred in, and the resulting mixture then stirred for 5 min, after which the stirring speed was reduced back to 100 rpm. The mixture was further stirred at 70° C. for 4 h, after which the mixture was transferred to an autoclave, in which the reaction mixture was heated to 150° C. and crystallized at that temperature under stirring for 168 h under autogenous pressure (measured pressure: 3.6 bar).

The resulting crystalline product was then filtered off under nitrogen atmosphere and then washed with 3.5 L distilled water and the solid dried under a nitrogen stream (10 m³/h) heated to 100° C. The resulting filter cake was then further dried at 120° C. for 16 h and then gradually heated to 500° C. during 500 min and then held at that temperature for 5 h for calcination, thus affording 4.751 kg of a beige crystalline powder.

According to the elemental analysis the resulting product had the following contents determined per 100 g substance of 0.008 g iron, 1.8 g aluminum, and 40.5 g silicon.

The BET surface area was determined to be 378 m²/g.

### Reference Example 3: Preparation of RUB-41 (RRO-Type Framework Structure)

5.8441 kg of an aqueous dimethyldipropylammonium hydroxide solution (41.73 wt-% in distilled water) were weighed into a 30 L vessel. 67.7 g of RUB-39 obtained according to WO 2005/100242 A1 were then added to the solution and the mixture was stirred for 10 min. 4.1059 kg of colloidal silica (Ludox AS 40) were then added under stirring, and the resulting mixture was then stirred for 1 h. The resulting suspension was placed in an autoclave and heated under autogenous pressure to 150° C. and held at that temperature for 48 h.

246.4 g of Al₂(SO₄)₃*18 H₂O were dissolved in 2.7359 kg of distilled water and stirred for 1 h. The solution was then added to the reaction mixture in the autoclave which was heated anew to 140° C. and held at that temperature for 72 h. The reaction product was then filtered and the solid product washed for obtained 703.4 g of a white powder.

150 g of the white powder were then heated to 600° C. using a ramp of 1° C./min and calcined under air at that temperature for 10 h for affording RUB-41.

According to the elemental analysis the resulting product had the following contents determined per 100 g substance of 1.9 g aluminum and 42 g silicon.

The BET surface area was determined to be 363 m²/g.

### Reference Example 4: Preparation of La-ZSM-5 by Wet Impregnation and Extrusion Thereof

50 g of H-ZSM-5 as obtained according to Reference Example 1 were added to a solution of 19.55 g of La(NO₃)₃*6 H₂O dissolved in 50 ml of distilled water and the mixture was stirred at room temperature for 2 h, after which the mixture was heated to 50° C. and evaporated to dryness over night in a rotary evaporator. The solid residue was then heated to 500° C. at a rate of 2° C./min and calcined at that temperature for 5 h for obtaining 57.8 g of La-ZSM-5.

The BET surface area was determined to be 278 m²/g.

The La-ZSM-5 was then admixed with 13.89 g of colloidal silica (Ludox AS-40) and 2.5 g Walocel binder (Wolf Walsrode AG PUFAS Werk KG), wherein the resulting mixture was kneaded for 10 min, after which 34 ml of distilled water were added and the resulting mixture was kneaded for an additional 20 min. The kneaded mixture was than extruded to strands with a diameter of 2 mm. The extrudate was then heated to 120° C. at a rate of 3° C./min, held at that temperature for 7 hours, and then heated further to 500° C. at a rate of 2° C./min and calcined at that temperature for 2 h for obtaining 37.3 g of the calcined extrudate.

### Reference Example 5: Preparation of La-ZSM-5 by Wet Impregnation and Extrusion Thereof

70 g of H-ZSM-5 as obtained according to Reference Example 1 were added to a solution of 13.69 g of La(NO₃)₃*6 H₂O dissolved in 70 ml of distilled water and the mixture was stirred at room temperature for 2 h, after which the mixture was heated to 50° C. and evaporated to dryness over night in a rotary evaporator. The solid residue was then heated to 500° C. at a rate of 2° C./min and calcined at that temperature for 5 h for obtaining 79.5 g of La-ZSM-5.

The BET surface area was determined to be 318 m²/g.

The La-ZSM-5 was then admixed with 22.08 g of colloidal silica (Ludox AS-40) and 3.9 g Walocel binder (Wolf Walsrode AG PUFAS Werk KG), wherein the resulting mixture was kneaded for 10 min, after which 50 ml of distilled water were added and the resulting mixture was kneaded for an additional 20 min. The kneaded mixture was than extruded to strands with a diameter of 2 mm. The extrudate was then heated to 120° C. at a rate of 3° C./min, held at that temperature for 7 hours, and then heated further to 500° C. at a rate of 2° C./min and calcined at that temperature for 2 h for obtaining 66.2 g of the calcined extrudate.

### Reference Example 6: Preparation of La-ZSM-5 by Wet Impregnation and Extrusion Thereof

50 g of H-ZSM-5 as obtained according to Reference Example 1 were added to a solution of 9.78 g of La(NO₃)₃*6 H₂O dissolved in 50 ml of distilled water and the mixture was stirred at room temperature for 2 h, after which the mixture was heated to 50° C. and evaporated to dryness over night in a rotary evaporator. The solid residue was then heated to 500° C. at a rate of 2° C./min and calcined at that temperature for 5 h for obtaining 52.67 g of La-ZSM-5.

The BET surface area was determined to be 322 m²/g.

50 g of the La-ZSM-5 were then admixed with 13.89 g of colloidal silica (Ludox AS-40) and 2.5 g Walocel binder (Wolf Walsrode AG PUFAS Werk KG), wherein the resulting mixture was kneaded for 10 min, after which 47 ml of distilled water were added and the resulting mixture was kneaded for an additional 20 min. The kneaded mixture was than extruded to strands with a diameter of 1.9 mm. The extrudate was then heated to 120° C. in 60 min, held at that temperature for 5 hours, and then heated further to 500° C. in 4 h and calcined in air at that temperature for 5 h for obtaining 43.7 g of the calcined extrudate.

### Reference Example 7: Preparation of La-ZSM-5 by Dry Impregnation and Extrusion Thereof

70 g of H-ZSM-5 as obtained according to Reference Example 1 were admixed with 26.97 g of La(NO₃)₃*6 H₂O, and the resulting mixture was ground in a laboratory mill (Microton; grinding at level 4) for 15 min. The ground mixture was then heated to 500° C. at a rate of 2° C./min and calcined at that temperature for 3 h for obtaining 79.4 g of La-ZSM-5

The BET surface area was determined to be 287 m²/g.

78 g of the La-ZSM-5 were then admixed with 21.66 g of colloidal silica (Ludox AS-40) and 3.9 g Walocel binder (Wolf Walsrode AG PUFAS Werk KG), wherein the resulting mixture was kneaded for 10 min, after which 50 ml of distilled water were added and the resulting mixture was kneaded for an additional 20 min. The kneaded mixture was than extruded to strands with a diameter of 2 mm. The extrudate was then heated to 120° C. at a rate of 3° C./min, held at that temperature for 7 hours, and then heated further to 500° C. at a rate of 2° C./min and calcined at that temperature for 2 h for obtaining 68.8 g of the calcined extrudate.

### Reference Example 8: Preparation of La-ZBM-10 by Wet Impregnation and Extrusion Thereof

80 g of ZBM-10 as obtained according to Reference Example 2 were added to a solution of 31.28 g of La(NO₃)₃*6 H₂O dissolved in 120 ml of distilled water and the mixture was stirred at room temperature for 2 h, after which the mixture was heated to 90° C. and evaporated to dryness over night in a rotary evaporator. The solid residue was then heated to 500° C. at a rate of 2° C./min and calcined at that temperature for 5 h for obtaining 89.6 g of La-ZBM-10.

The BET surface area was determined to be 238 m²/g.

The La-ZBM-10 was then admixed with 55.63 g of colloidal silica (Ludox AS-40) and 4.45 g Walocel binder (Wolf Walsrode AG PUFAS Werk KG), wherein the resulting mixture was kneaded for 10 min, after which 62 ml of distilled water were added and the resulting mixture was kneaded for an additional 35 min. The kneaded mixture was than extruded to strands with a diameter of 1.5 mm. The extrudate was then heated to 120° C. at a rate of 3° C./min, held at that temperature for 7 hours, and then heated further to 500° C. at a rate of 2° C./min and calcined at that temperature for 2 h for obtaining 89.4 g of the calcined extrudate.

### Reference Example 9: Preparation of Ce-ZSM-5 by Wet Impregnation and Extrusion Thereof

70 g of H-ZSM-5 as obtained according to Reference Example 1 were added to a solution of 26.81 g of Ce(NO₃)₃*6 H₂O dissolved in 70 ml of distilled water and the mixture was stirred at room temperature for 2 h, after which the mixture was heated to 50° C. and evaporated to dryness over night in a rotary evaporator. The solid residue was then heated to 500° C. at a rate of 2° C./min and calcined at that temperature for 5 h for obtaining 80.5 g of Ce-ZSM-5.

The BET surface area was determined to be 336 m²/g.

The Ce-ZSM-5 was then admixed with 22.36 g of colloidal silica (Ludox AS-40) and 4.03 g Walocel binder (Wolf Walsrode AG PUFAS Werk KG), wherein the resulting mixture was kneaded for 10 min, after which 54 ml of distilled water were added and the resulting mixture was kneaded for an additional 20 min. The kneaded mixture was than extruded to strands with a diameter of 2 mm. The extrudate was then heated to 120° C. at a rate of 3° C./min, held at that temperature for 7 hours, and then heated further to 500° C. at a rate of 2° C./min and calcined at that temperature for 2 h for obtaining 66.5 g of the calcined extrudate.

### Comparative Example 1: Preparation of La-RUB-41 by Wet Impregnation and Extrusion Thereof

60 g of RUB-41 as obtained according to Reference Example 3 were added to a solution of 23.46 g of La(NO₃)₃*6 H₂O dissolved in 120 ml of distilled water and the mixture was stirred at room temperature for 2 h, after which the mixture was heated to 90° C. and evaporated to dryness over night in a rotary evaporator. The solid residue was then heated to 500° C. at a rate of 2° C./min and calcined at that temperature for 5 h for obtaining 66.7 g of La-RUB-41.

The BET surface area was determined to be 201 m²/g.

66 g of the La-RUB-41 were then admixed with 41.25 g of colloidal silica (Ludox AS-40) and 3.3 g Walocel binder (Wolf Walsrode AG PUFAS Werk KG), wherein the resulting mixture was kneaded for 10 min, after which 30 ml of distilled water were added and the resulting mixture was kneaded for an additional 20 min. The kneaded mixture was than extruded to strands with a diameter of 2 mm. The extrudate was then heated to 120° C. at a rate of 3° C./min, held at that temperature for 7 hours, and then heated further to 500° C. at a rate of 2° C./min and calcined at that temperature for 2 h for obtaining 61.1 g of the calcined extrudate.

### Comparative Example 2: Preparation of Extrudates with ZSM-5

60 g of H-ZSM-5 as obtained according to Reference Example 1 were admixed with 16.66 g of colloidal silica (Ludox AS-40) and 3 g Walocel binder (Wolf Walsrode AG PUFAS Werk KG), wherein the resulting mixture was kneaded for 10 min, after which 50 ml of distilled water were added and the resulting mixture was kneaded for an additional 20 min. The kneaded mixture was than extruded to strands with a diameter of 1 mm. The extrudate was then heated to 120° C. at a rate of 3° C./min, held at that temperature for 7 hours, and then heated further to 500° C. at a rate of 2° C./min and calcined at that temperature for 2 h for obtaining 48.4 g of the calcined extrudate.

### Comparative Example 3: Preparation of Extrudates with ZBM-10 (MEL/MFI Intergrowth)

100 g of ZBM-10 as obtained according to Reference Example 2 were admixed with 27.78 g of colloidal silica (Ludox AS-40) and 5 g Walocel binder (Wolf Walsrode AG PUFAS Werk KG), wherein the resulting mixture was kneaded for 10 min, after which 120 ml of distilled water were added and the resulting mixture was kneaded for an additional 35 min. The kneaded mixture was than extruded to strands with a diameter of 1.5 mm. The extrudate was then heated to 120° C. at a rate of 3° C./min, held at that temperature for 7 hours, and then heated further to 500° C. at a rate of 2° C./min and calcined at that temperature for 2 h for obtaining 89.5 g of the calcined extrudate.

### Comparative Example 4: Preparation of La—Al₂O₃/SiO₂ by Wet Impregnation and Extrusion Thereof

A support which is commercially available from BASF SE (product name: D10-10) consisting of about 100 weight-% of silica and alumina was provided, wherein the weight ratio of silica relative to alumina was about 1:4. The support had a pore volume of 0.58 cm³/g and an acidity characterized by an amount of adsorbed ammonia of 0.5 mmol/g. The support was in the form of extrudates having an essentially circular cross-section with a diameter of 2 mm.

100 g of the support were added to a solution of 38.53 g of La(NO₃)₃*6 H₂O dissolved in 100 ml of distilled water and the mixture was stirred at room temperature for 2 h, after which the mixture was heated to 50° C. and evaporated to dryness over night in a rotary evaporator. The solid residue was then heated to 500° C. at a rate of 2° C./min and calcined at that temperature for 5 h for obtaining 95 g of La—Al₂O₃/SiO₂.

The BET surface area was determined to be 347 m²/g.

The La—Al₂O₃/SiO₂ was then admixed with 26.39 g of colloidal silica (Ludox AS-40) and 4.75 g Walocel binder (Wolf Walsrode AG PUFAS Werk KG), wherein the resulting mixture was kneaded for 10 min, after which 65 ml of distilled water were added and the resulting mixture was kneaded for an additional 20 min. The kneaded mixture was than extruded to strands with a diameter of 1.5 mm. The extrudate was then heated to 120° C. at a rate of 3° C./min, held at that temperature for 7 hours, and then heated further to 500° C. at a rate of 2° C./min and calcined at that temperature for 2 h for obtaining 78.1 g of the calcined extrudate.

### Comparative Example 5: Preparation of La-ZSM-5 by Ion Exchange

The procedure described in the experimental section of U.S. Pat. No. 5,999,999 for obtaining the “Catalyst A” described therein was repeated. To this effect, 50 g of H-ZSM-5 as obtained according to Reference Example 1 were added to a solution of 216.5 g of La(NO₃)₃*6 H₂O dissolved in 500 ml of distilled water (1 M lanthanum nitrate solution) and the mixture was stirred at room temperature for 24 h. The solid was then filtered off and washed with 4 L of distilled water, after which is was dried at 100° C. for 24 h for affording 51.3 g of La-ZSM-5.

According to the elemental analysis the resulting product had the following contents determined per 100 g substance of 1.0 g lanthanum, 1.5 g aluminum, and 40 g silicon.

Accordingly, repetition of the procedure from U.S. Pat. No. 5,999,999 reveals that the product displays a loading of 1 wt.-% of lanthanum as opposed to 10 wt.-% as indicated U.S. Pat. No. 5,999,999.

### Examples: Amination of Ethylene Oxide

The extrudated material from the respective reference and comparative examples was filtered for obtaining a split fraction in the range of from 0.4-0.8 mm, which was then filed into the reactor (tubular reactor with a length of 1350 mm and a diameter of 0.5 mm (reactor volume=3.66 ml/m), wherein the reactor had a wall thickness of 3.17 mm), and the reactor vessel was then flooded with nitrogen prior to testing.

Ethylene oxide and ammonia were continually pumped into a pre-mixing unit (2.0 ml volume) and then introduced into the reactor which was heated to a given temperature for reacting the mixture over the catalyst sample. For the analysis of the product mixture, a sample of 0.25 ml was collected and was quenched in a pressure vessel with HOAc (7.0 ml). For analytical assessment, 0.75 ml of the sample were then transferred to a gas chromatography-phial and then tempered for 16 h at 65° C., after which 0.75 ml of Ac₂O were added and the sample incubated at 65° C. for an additional 16 h. The gas chromatographical analysis was conducted on a 60 m RTX1 column (temperature ramp: 80° C. starting temperature and heating at a rate of 8° C./min to 280° C.) with the following retention times: rₜ (MEOA)=16.66 min; rₜ (DEOA)=23.96 min; rₜ (TEOA)=25.21 min.

TABLE 1
Results from the amination of ethylene oxide at a NH3:EO molar ratio of
~21 using the catalysts from Reference Examples 4-9 and Comparative
Examples 1-4 for different temperature ranges (40, 59-60, 66-73, 90-93,
109-110) including the Lewis and Bronsted acidities of the zeolitic materials.

| | | Metal | Lewis | Bronsted | Temp. | Conv. | Product distribution | | |
|---|---|---|---|---|---|---|---|---|---|
| Catalyst | framework | (wt.-%) | acidity | acidity | [° C.] | [%] | MEOA | DEOA | TEOA |
| RE 4 | MFI | La (9.4) | 87.68 | 17.05 | 110 | 99.8 | 75.3 | 24.5 | 0.2 |
| RE 7 | MFI | La (9.1) | 78.96 | 20.4 | 110 | 99.9 | 72.7 | 27.1 | 0.1 |
| RE 9 | MFI | Ce (8.7) | 86.7 | 10.8 | 110 | 99.9 | 73.5 | 26.3 | 0.2 |
| RE 8 | MEL/MFI | La (9) | 81.9 | 14.8 | 110 | 100 | 75.5 | 24.5 | 0 |
| CE 2 | MFI | — | 75.2 | 39.6 | 110 | 50.0 | 84.4 | 14.4 | 1.2 |
| CE 2 | MFI | — | 75.2 | 39.6 | 110 | 44.7 | 87.6 | 11.6 | 0.8 |
| CE 3 | MEL/MFI | — | 49 | 21.6 | 110 | 54.1 | 82.3 | 16.7 | 1.0 |
| CE 1 | RRO | La (11) | n.a. | n.a. | 110 | 50.1 | 89.1 | 10.0 | 0.9 |
| CE 4 | — | La (11.3) | 77.95 | 0 | 109 | 18.5 | 88.3 | 8.9 | 2.8 |
| RE 4 | MFI | La (9.4) | 87.68 | 17.05 | 90 | 99.9 | 78.8 | 21.3 | 0.0 |
| RE 7 | MFI | La (9.1) | 78.96 | 20.4 | 93 | 99.9 | 75.9 | 24 | 0.1 |
| CE 2 | MFI | — | 75.2 | 39.6 | 90 | 20.9 | 93.5 | 6.0 | 0.6 |
| CE 3 | MEL/MFI | — | 49 | 21.6 | 90 | 29.5 | 98.6 | 9.6 | 0.8 |
| CE 1 | RRO | La (11) | n.a. | n.a. | 90 | 11.7 | 98.7 | 1.3 | 0.0 |
| RE 6 | MFI | La (5.1) | 83.9 | 22.1 | 66 | 100 | 78.8 | 21.2 | 0 |
| RE 5 | MFI | La (4.9) | 86.9 | 18.7 | 70 | 99.5 | 77.7 | 22.7 | 0 |
| RE 9 | MFI | Ce (8.7) | 86.7 | 10.8 | 73 | 51.1 | 87.6 | 12.2 | 0.2 |
| CE 4 | — | La (11.3) | 77.95 | 0 | 70 | 6.3 | 97.2 | 1.8 | 1.2 |
| RE 5 | MFI | La (4.9) | 86.9 | 18.7 | 59 | 99.1 | 78.2 | 21.7 | 0 |
| RE 8 | Mel/MFI | La (9) | 81.9 | 14.8 | 60 | 84.8 | 83 | 17.05 | 0 |
| RE 9 | MFI | Ce (8.7) | 86.7 | 10.8 | 60 | 25 | 94.6 | 12.2 | 0.2 |
| RE 5 | MFI | La (4.9) | 86.9 | 18.7 | 40 | 84.6 | 82.8 | 17.2 | 0 |
| RE 8 | Mel/MFI | La (9) | 81.9 | 14.8 | 40 | 47.3 | 89.6 | 10.3 | 0 |

TABLE 2
Results from the amination of ethylene oxide at a NH3:EO molar ratio of ~21
using the catalysts from Reference Examples 4-9 and Comparative Examples 1-4
for different temperature ranges (40, 59-60, 66-73, 90-93, 109-110; see Table 1) including
the acidities of the zeolitic materials as determined from NH₃-TPD.

| | | | Total | Weak | Medium | Strong | | | |
|---|---|---|---|---|---|---|---|---|---|
| | | | Acid | Acid | Acid | Acid | | | |
| | | | Sites | Sites | Sites | Sites | | | |
| | | Metal | [mmol/ | [mmol/ | [mmol/ | [mmol/ | Product distribution | | |
| Catalyst | framework | (wt.-%) | g] | g] | g] | g] | MEOA | DEOA | TEOA |
| RE 4 | MFI | La (9.4) | 0.682 | 0.46 | 0.22 | 0 | 75.3 | 24.5 | 0.2 |
| RE 7 | MFI | La (9.1) | 0.57 | 0.33 | 0.23 | 0.008 | 72.7 | 27.1 | 0.1 |
| RE 9 | MFI | Ce (8.7) | 0.697 | 0.379 | 0.318 | 0 | 73.5 | 26.3 | 0.2 |
| RE 8 | MEL/MFI | La (9) | 0.553 | 0.399 | 0.151 | 0.003 | 75.5 | 24.5 | 0 |
| CE 2 | MFI | — | 0.741 | 0.38 | 0.37 | 0 | 84.4 | 14.4 | 1.2 |
| CE 2 | MFI | — | 0.741 | 0.38 | 0.37 | 0 | 87.6 | 11.6 | 0.8 |
| CE 3 | MEL/MFI | — | 0.67 | 0.36 | 0.3 | 0.007 | 82.3 | 16.7 | 1.0 |
| CE 1 | RRO | La (11) | 0.246 | 0.201 | 0 | 0.045 | 89.1 | 10.0 | 0.9 |
| CE 4 | — | La (11.3) | 0.399 | 0.327 | 0 | 0.072 | 88.3 | 8.9 | 2.8 |
| RE 4 | MFI | La (9.4) | 0.682 | 0.46 | 0.22 | 0 | 78.8 | 21.3 | 0.0 |
| RE 7 | MFI | La (9.1) | 0.57 | 0.33 | 0.23 | 0.008 | 75.9 | 24 | 0.1 |
| CE 2 | MFI | — | 0.741 | 0.38 | 0.37 | 0 | 93.5 | 6.0 | 0.6 |
| CE 3 | MEL/MFI | — | 0.67 | 0.36 | 0.3 | 0.007 | 98.6 | 9.6 | 0.8 |
| CE 1 | RRO | La (11) | 0.246 | 0.201 | 0 | 0.045 | 98.7 | 1.3 | 0.0 |
| RE 6 | MFI | La (5.1) | 0.553 | 0.399 | 0.151 | 0.003 | 78.8 | 21.2 | 0 |
| RE 5 | MFI | La (4.9) | 0.677 | 0.418 | 0.255 | 0.004 | 77.7 | 22.7 | 0 |
| RE 9 | MFI | Ce (8.7) | 0.697 | 0.379 | 0.318 | 0 | 87.6 | 12.2 | 0.2 |
| CE 4 | — | La (11.3) | 0.399 | 0.327 | 0 | 0.072 | 97.2 | 1.8 | 1.2 |
| RE 5 | MFI | La (4.9) | 0.677 | 0.418 | 0.255 | 0.004 | 78.2 | 21.7 | 0 |
| RE 8 | Mel/MFI | La (9) | 0.553 | 0.399 | 0.151 | 0.003 | 83 | 17.05 | 0 |
| RE 9 | MFI | Ce (8.7) | 0.697 | 0.379 | 0.318 | 0 | 94.6 | 12.2 | 0.2 |
| RE 5 | MFI | La (4.9) | 0.677 | 0.418 | 0.255 | 0.004 | 82.8 | 17.2 | 0 |
| RE 8 | Mel/MFI | La (9) | 0.553 | 0.399 | 0.151 | 0.003 | 89.6 | 10.3 | 0 |

Thus, as may be taken from the results displayed in Table 1, compared to the comparative examples, the catalysts of the invention lead to substantially higher conversion rates, wherein practically complete conversion of ethylene oxide may be achieved for selected inventive catalysts at temperatures as low as 59° C. (see results using catalyst from Reference Example 5). This is in clear contrast to catalysts which either do not contain any rare earth metals, or for those not containing a zeolite (see results using catalyst from Comparative Example 4 with Al₂O₃) or containing a zeolite with a different framework-type structure than the inventive catalysts (see results using catalyst from Comparative Example 1). Same applies accordingly with regard to the production of Tri(2-hydroxyethyl)amine (TEOA), which may be substantially reduced or practically eliminated compared to the comparative examples. In addition to the aforementioned, it has quite unexpectedly been found that these surprising effects which may be achieved with the inventive process does not jeopardize the selectivity towards the desired products 2-aminoethanol (MEOA) and Di(2-hydroxyethyl)amine (DEOA), wherein in particular very high selectivities towards 2-aminoethanol may be achieved using the inventive process.

It has also surprisingly been found that lanthanum offers the best results with regard to the conversion rates which may be achieved at lower temperatures, e.g. compared to the use of cerium (see results using catalyst from Reference Example 9), such that the use of lanthanum as the rare earth metal in the catalyst of the inventive process is particularly preferred.

Consequently, it has unexpectedly been found that a substantially improved process for the amination of alkylene oxides may be provided according to the present invention, in particular with regard to both the conversion rate and the selectivities towards the desired products, wherein the surprising technical effects of the inventive process are particularly pronounced at lower reaction temperatures.

### LIST OF CITED DOCUMENTS

DE 1941859
U.S. Pat. No. 3,697,598
U.S. Pat. No. 4,438,281
EP 0375267 A2
CN 101884934
Feng, R. et al. in Catalysis Communications 2010, 11, pp. 1220-1223
U.S. Pat. No. 5,599,999
U.S. Pat. No. 6,169,207 B1
Pouria, R. et al. in Journal of Rare Earths 2017, 35, 542-550
EP 1 104 752 A2
JP 2002 028492 A
EP 1 219 592 A1
Eric Marceau et al. in “Ion Exchange and Impregnation: “Handbook of heterogeneous catalysis” (1972), vol. 107, pages 467-484

1.-15. (canceled)

16. A process for the conversion of ethylene oxide to 2-aminoethanol and/or Di(2-hydroxyethyl)amine comprising
(i) providing a catalyst comprising a zeolitic material comprising YO₂ and X₂O₃ in its framework structure, wherein Y is a tetravalent element and X is a trivalent element, wherein the zeolitic material has a framework-type structure selected from the group consisting of MFI and/or MEL, including MEL/MFI intergrowths, and wherein the zeolitic material contains one or more rare earth elements;
(ii) providing a mixture in the liquid phase comprising ethylene oxide and ammonia;
(iii) contacting the catalyst provided in (i) with the mixture in the liquid phase provided in (ii) for converting ethylene oxide to 2-aminoethanol and/or Di(2-hydroxyethyl)amine, wherein the catalyst provided in (i) is obtained and/or obtainable by a process comprising loading one or more salts of the one or more rare earth elements into the pores of the porous structure of the zeolitic material and optionally on the surface of the zeolitic material.

17. The process of claim 16, wherein Y is selected from the group consisting of Si, Sn, Ti, Zr, Ge, and mixtures of two or more thereof.

18. The process of claim 16, wherein X is selected from the group consisting of Al, B, In, Ga, and mixtures of two or more thereof.

19. The process of claim 16, wherein the one or more rare earth elements are selected from the group consisting of
Ce, Dy, Er, Eu, Gd, Ho, La, Lu, Nd, Pr, Pm, Sm, Sc, Tb, Tm, Yb, and Y.

20. The process of claim 16, wherein the RE:X₂O₃ molar ratio of the one or more rare earth elements to X₂O₃ contained in the framework structure of the zeolitic material is in the range of from 0.1 to 6.

21. The process of claim 16, wherein the zeolitic material contains substantially no Na.

22. The process of claim 16, wherein the catalyst provided in (i) and contacted with the mixture in the liquid phase in (iii) displays an amount of strong acid sites as determined by NH₃-TPD of 0.05 mmol/g or less.

23. The process of claim 16, wherein the molar ratio of weak acid sites to medium acid sites as respectively determined by NH₃-TPD of the catalyst provided in (i) and contacted with the mixture in the liquid phase in (iii) is in the range of from 0.1 to 5.

24. The process of claim 16, wherein the loading of the one or more salts of the one or more rare earth elements into the pores of the porous structure of the zeolitic material and optionally on the surface of the zeolitic material comprises
(a) impregnating the porous structure of the zeolitic material with a solution of the one or more salts of the one or more rare earth elements;
(b) optionally drying the impregnated zeolitic material obtained in (b);
(c) calcining the zeolitic material obtained in (a) or (b).

25. The process of claim 24, wherein the volume of the solution employed in (a) is equal to 500% or less of the total pore volume of the zeolitic material prior to impregnation with the solution, wherein the total pore volume is determined by nitrogen adsorption from the BJH method.

26. The process of claim 16, wherein the loading of the one or more salts of the one or more rare earth elements into the pores of the porous structure of the zeolitic material and optionally on the surface of the zeolitic material comprises
(a′) preparing a mixture of the one or more salts of the one or more rare earth elements and the zeolitic material;
(b′) optionally milling the mixture obtained in (a′);
(c′) calcining the zeolitic material obtained in (a′) or (b′).

27. The process of claim 24, wherein prior to the loading of the one or more salts of the one or more rare earth elements into the pores of the porous structure of the zeolitic material and optionally on the surface of the zeolitic material in (a) or (a′), the zeolitic material is in the H-form and contains protons as extra-framework ions, wherein 0.1 wt.-% or less of the extra-framework ions are metal cations, calculated as the element and based on 100 wt.-% of YO₂ contained in the zeolitic material.

28. The process of claim 16, wherein the catalyst provided in (i) and contacted with the mixture in the liquid phase in (iii) is obtained and/or obtainable by a process which does not comprise a step of ion exchanging the one or more rare earth elements into the zeolitic material.

29. The process of claim 16, wherein the contacting in (iii) is effected at a temperature in the range of from 40 to 180° C.

30. The process of claim 16, wherein the contacting in (iii) is effected at a pressure in the range of from 50 to 250 bar.
